(19) United States
(12) Patent Application Publication
(10) Pub. No.: US 2025/0146694 A1
(43) Pub. Date: May 8, 2025

(54) METHOD FOR CONTROLLING AN AIR CONDITIONER IN A MULTI-USER ENVIRONMENT, AND THE AIR CONDITIONER

(71) Applicant: SAMSUNG ELECTRONICS CO., LTD., Suwon-si (KR)

(72) Inventor: Choice CHOUDHARY, Haryana (IN)

(73) Assignee: SAMSUNG ELECTRONICS CO., LTD., Suwon-si (KR)

(21) Appl. No.: 19/020,499

(22) Filed: Jan. 14, 2025

Related U.S. Application Data
(63) Continuation of application No. PCT/KR2023/006225, filed on May 8, 2023.

(30) Foreign Application Priority Data
Sep. 9, 2022 (IN) 202211051786

(51) Int. Cl. F24F 11/63

(52) U.S. Cl. CPC F24F 11/63

(57) ABSTRACT

A method for controlling an air conditioner in a multi-user environment includes identifying one or more activities of each of a plurality of users; obtaining one or more physiological parameters of each of a plurality of users and one or more environment conditions; creating a profile for each of the plurality of users based on at least one of the one or more physiological parameters or one or more environment conditions; generating a composite profile for the plurality of users; and modulating a setting of the air conditioner based on the generated composite profile of each of the plurality of users.

### CROSS-REFERENCE TO RELATED APPLICATIONS

This application is a continuation of International Application No. PCT/KR2023/006225, filed on May 8, 2023, which is based on and claims priority to Indian Patent Application number 202211051786, filed on Sep. 9, 2022, in the Indian Patent Office, the disclosures of which are incorporated by reference herein in their entireties.

### BACKGROUND

### 1. Field

The present disclosure relates to a method of controlling an air conditioner and, more particularly, to a method for controlling the air conditioner in a multi-user environment, and the air conditioner.

### 2. Description of the Related Art

Due to climate change, demand for cooling an indoor environment has significantly increased globally. At present, cooling of the indoor environment can be achieved by utilizing an air conditioner and climate control system. The air conditioner enables people from every corner of the world to live their lives in a proper microclimatic environment and therefore is seen to be utilized in many different places, including homes, offices, malls, or in any other closed area. The air conditioner engages processes such as cooling, heating, sterilization, humidification, and dehumidification of the air. In today's world, people cannot imagine their lives without air conditioners because they feel uncomfortable without air conditioners.

The technology that goes into manufacturing the air conditioner has come a long way since its inception. With the advancement in technology, the air conditioner has now become smaller and more efficient. The air conditioner comes with built-in sensors that help in determining the required temperature of the indoor environment. Further, there are some more advanced features, one of the advanced features that is commonly found in the air conditioner is remote control operation for adjusting the air conditioning (ac) mode and fan speed without having to physically move to adjust a the air conditioning (ac) mode and fan speed. Another useful feature includes enabling the bio-sleep functionality to create a comfortable sleeping environment for the user.

The bio-sleep functionality is a special feature that adjusts the temperature automatically. Since the human body is incapable of handling cooling during the sleep state and therefore the bio-sleep functionality regulates the ambient temperature much better without anyone noticing it. Further, this feature maintains optimal body temperature for maximum rest and reduces energy consumption by some percentage (approximately 36%) compared to conventional cooling mode, and also improves the air conditioner's health.

Current air conditioner control methods by a wearable device includes receiving the sleep quality information of the user detected by the wearable device. The sleep quality information includes deep sleep information, shallow sleep status information, and awake state information. Sleep parameters value of the air-conditioner may be adjusted according to sleep quality information and default user type. The air-conditioner operation may be controlled according to the sleep parameters value after its adjustment. The temperature and wind speed output of the air-conditioner may be adjusted in real time according to the sleep quality information and the type of user in order to promote the sleep quality of different types of users and improve user's comfort. However, the current methods fail to receive one or more sleep states of a plurality of users and determining the required air conditioner temperature based upon properties of the one or more sleep states by using artificial intelligence. Further, the current methods fail to identify the number of a plurality of users in the indoor environment and determining the temperature sensitivity of each of the plurality of users. Also, the current methods are not applicable to the plurality of users who do not have a wearable device while sleeping. In addition, the current methods fail to provide a set of recommendations to each of the plurality of users for improving their sleep scores without impacting the sleep scores of other users.

Current climate control mattress systems provide desired climate conditions of temperature and humidity at a contact surface of a multi-layered structure. The climate control system includes a heating mechanism disposed between one or more foam layers of structure and configured to deliver heat to the contact surface and a separate cooling mechanism disposed in the foam layers. The cooling mechanism includes at least one fan assembly, air channels, and reticulated foam layers configured to draw air away from the contact surface toward the bottom of the structure. An operational control system is used to control the heating mechanism and cooling mechanisms to achieve the desired climate conditions on the contact surface in accordance with a desired time-based climate control algorithm. However, the current climate control mattress systems fail to disclose receiving one or more sleep states of a plurality of users and determining the required air conditioner temperature based upon the properties of one or more sleep states by using artificial intelligence. Further, the current climate control mattress systems fail to identify the number of a plurality of users in the indoor environment and determining the temperature sensitivity of each of the plurality of users. Also, the current climate control mattress systems are not applicable to the plurality of users who do not have a wearable device while sleeping. In addition, current climate control mattress systems fail to provide a set of recommendations to each of the plurality of users for improving their sleep scores without impacting the sleep scores of other users.

Therefore, there exists a need to overcome the aforementioned drawbacks associated with the existing method and system for enabling the bio-sleep functionality of the device in a multi-user environment.

### SUMMARY

Additional aspects will be set forth in part in the description which follows and, in part, will be apparent from the description, or may be learned by practice of the presented embodiments.

According to an aspect of the disclosure, a method for controlling an air conditioner in a multi-user environment includes identifying, by one or more sensors, one or more activities of each of a plurality of users; obtaining, by the one or more sensors, one or more physiological parameters of each of the plurality of users and one or more environment conditions; creating, by one or more processors, a profile for each of the plurality of users based on at least one of the one or more physiological parameters or the one or more environment conditions; generating, by the one or more processors, a composite profile for each of the plurality of users; and modulating, by the one or more processors, a setting of the air conditioner based on the composite profile of each of the plurality of users.

The one or more activities may include active state and sleep state activity of one of the plurality of users. The one or more physiological parameters may include at least one of heart rate, stress level, blood pressure, SPO2 level, or body temperature of one of the plurality of users. The one or more environment conditions may include at least one of ambient temperature, humidity, or sleep timing pattern.

The creating the profile for each of the plurality of users may include: determining, by the one or more processors, sleep quality of each of the plurality of users in one or more sleep states using one or more derived parameters, wherein the one or more derived parameters comprise at least one of a sleeping posture, sleep waking, dehydration, or frequency of change in the sleeping posture; optimizing, by the one or more processors, a duration of the one or more sleep states based on the one or more sleep states and minimizing power consumption by modulating the setting, wherein the setting comprises one or more air conditioning (ac) modes, fan speeds, and ac temperature; and creating, by the one or more processors, the profile for each of the plurality of users based on at least one of the determined sleep quality, the optimized duration of the one or more sleep states, or the modulated setting.

The generating the composite profile for each of the plurality of users may include: monitoring, by the one or more sensors, the one or more activities of each of the plurality of users; enabling, by the one or more processors, a bio-sleep functionality; identifying, by the one or more sensors or one or more electronic devices, a number of the plurality of users present in the multi-user environment and classifying each of the plurality of users; receiving, by the one or more processors, the one or more physiological parameters of each of the plurality of users; determining, by the one or more processors, a priority score of each of the plurality of users based on a first set of parameters received from the profile; determining, by the one or more processors, the setting of the air conditioner based on an aggregated setting for all of the plurality of users, wherein the aggregated setting comprises the setting for all of the plurality of users received from the profile; determining, by the one or more processors, a collective sleep quality score based on a sleep quality score of each of the plurality of users for the setting for all of the plurality of users received from the profile; enabling, by the one or more processors, the setting of the air conditioner based on determining a highest collective sleep quality score of the sleep quality score of each of the plurality of users; and generating, by the one or more processors, the composite profile based on the setting for all of the plurality of users received from the profile.

The bio-sleep functionality may be enabled based on the one or more physiological parameters including blood viscosity and body temperature of each of the plurality of users, the one or more environment conditions including humidity, and a status of the air conditioner including backup power connection.

The one or more sensors may include at least one of a camera sensor or a motion sensor mounted on the air conditioner. The one or more electronic devices may be connected to the air conditioner via Bluetooth technology or Wi-Fi technology. The method may further include classifying, by the one or more processors, each of the plurality of users into including first category of users with wearable devices and a second category of users without wearable devices.

The receiving of the one or more physiological parameters of each of the plurality of users may include: receiving, from the wearable devices, the one or more physiological parameters of each of the plurality of users with the wearable devices; and receiving, from electronic devices, the one or more physiological parameters of each of the plurality of users without the wearable devices, the electronic devices being at least one of a mobile phone, PDA, or ac remote control. The wearable devices may include at least one of wearable electronic devices, electronic devices implanted in a user's body, wearable devices tattooed on skin, wristbands, or wristwatches. The wearable devices may be connected to the electronic devices by at least one of Bluetooth technology or Wi-Fi technology.

The first set of parameters may include at least one of age, body mass index (BMI), sleep state sensitivity, current sleep state, or a duration of the current sleep state.

The aggregated setting may include: a preferred ac mode from one or more ac modes which comprising a dry mode, a fan mode, a cool mode, or sleep mode; a preferred fan speed from one or more fan speeds comprising at least one of a low speed, a medium speed, or high speed; and a preferred ac temperature being a sum of a product of the ac temperature received from the profile of each of the plurality of users and the priority score of each of the plurality of users.

The sleep quality score may be based on the preferred ac temperature, the preferred ac mode, the one or more derived parameters, and the frequency of change in the sleeping posture and the one or more physiological parameters.

According to another aspect of the disclosure, an air conditioner in a multi-user environment includes: one or more sensors configured to: identify one or more activities of each of a plurality of users; and obtain one or more physiological parameters of each of the plurality of users and one or more environment conditions; and one or more processors configured to: create a profile for each of the plurality of users based on at least one of the one or more physiological parameters and the one or more environment conditions; generate a composite profile for each of the plurality of users; and modulate a setting of the air conditioner based on the composite profile of each of the plurality of users.

The one or more activities may include active state and sleep state activity of one of the plurality of users. The one or more physiological parameters may include at least one of heart rate, stress level, blood pressure, SPO2 level, or body temperature of one of the plurality of users. The one or more environment conditions may niclude at least one of ambient temperature, humidity, or sleep timing pattern.

The one or more processors may be further configured to: determine sleep quality of each of the plurality of users in one or more sleep states using one or more derived parameters, wherein the one or more derived parameters comprise at least one of a sleeping posture, sleep waking, dehydration, or frequency of change in the sleeping posture; optimize a duration of the one or more sleep states based on the one or more sleep states and minimize power consumption by modulating the setting, wherein the setting comprises one or more air conditioning (ac) modes, fan speeds, and ac temperature; and create the profile for each of the plurality of users based on at least one of the determined sleep quality, optimized duration of the one or more sleep states, or the modulated setting.

The one or more sensors may be further configured to monitor the one or more activities of each of plurality of users. The one or more processors may be further configured to: enable a bio-sleep functionality; identify a number of the plurality of users present in the multi-user environment, classifying each of the plurality of users; receive the one or more physiological parameters of each of the plurality of users; determine a priority score of each of the plurality of users based on a first set of parameters received from the profile; determine the setting of the air conditioner based on an aggregated setting for all of the plurality of users, wherein the aggregated setting comprises the setting for all of the plurality of users received from the profile; determine a collective sleep quality score based on a sleep quality score of each of the plurality of users for the setting for all of the plurality of users received from the profile; enable the setting of the air conditioner based on a highest collective sleep quality score of the sleep quality score of each of the plurality of users; and generate the composite profile based on the setting for all of the plurality of users received from the profile.

The one or more processors may be further configured to enable the bio-sleep functionality based on the one or more physiological parameters including blood viscosity and body temperature of each of the plurality of users, the one or more environment conditions including humidity, and a status of the air conditioner including backup power connection.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 illustrates a flow diagram showing a method for enabling bio-sleep functionality of a device in a multi-user environment, in accordance with one or more embodiments of the present disclosure;

FIG. 2 illustrates a block diagram of an air conditioner in a multi-user environment, in accordance with one or more embodiments of the present disclosure;

FIG. 3 illustrates a flow diagram showing a method of creating a profile by a training phase module during the training phase of the device, in accordance with one or more embodiments of the present disclosure;

FIG. 4 illustrates a block diagram of the training phase module, in accordance with one or more embodiments of the present disclosure;

FIG. 5a illustrates a graphical representation of the variation of sleep quality with respect to frequency of change in sleeping posture, in accordance with one or more embodiments of the present disclosure;

FIG. 5b illustrates a graphical representation of the variation of sleep quality with respect to urination, in accordance with one or more embodiments of the present disclosure;

FIG. 5c illustrates a graphical representation of the variation of sleep quality with respect to change in blood pressure, in accordance with one or more embodiments of the present disclosure;

FIG. 5d illustrates a graphical representation of the variation of sleep quality with respect to SPO2 level, in accordance with one or more embodiments of the present disclosure;

FIG. 5e illustrates a graphical representation of the variation of sleep quality with respect to body temperature, in accordance with one or more embodiments of the present disclosure;

FIG. 5f illustrates a block diagram of sleeping posture for sleep quality determination, in accordance with one or more embodiments of the present disclosure;

FIG. 5g illustrates a pictorial representation of different sleeping postures, in accordance with one or more embodiments of the present disclosure;

FIG. 6 illustrates a block diagram of an optimization sub-module of the training phase module, in accordance with one or more embodiments of the present disclosure;

FIG. 7 illustrates a flow diagram showing a method of generating a composite profile by an application phase module in the multi-user environment, in accordance with one or more embodiments of the present disclosure;

FIG. 8a illustrates a block diagram of the application phase module, in accordance with one or more embodiments of the present disclosure;

FIG. 8b illustrates a graphical representation of the priority score with respect to the age, in accordance with one or more embodiments of the present disclosure;

FIG. 8c illustrates a graphical representation of the priority score with respect to the body mass index (BMI), in accordance with one or more embodiments of the present disclosure;

FIG. 8d illustrates a graphical representation of the priority score with respect to the sleep state sensitivity, in accordance with one or more embodiments of the present disclosure;

FIG. 8e illustrates a graphical representation of the priority score with respect to the current sleep state, in accordance with one or more embodiments of the present disclosure;

FIG. 8f illustrates a graphical representation of the priority score with respect to the duration of the current sleep state, in accordance with one or more embodiments of the present disclosure; and

FIG. 9 illustrates a diagram of the air conditioner according to one or more embodiments.

### DETAILED DESCRIPTION

In the following description, numerous specific details are set forth in order to provide a thorough understanding of the present disclosure. It will be apparent, however, to one skilled in the art that these specific details are only exemplary and not intended to be limiting. Additionally, it may be noted that the systems and/or methods are shown in block diagram form only in order to avoid obscuring the present disclosure. It is to be understood that various omissions and substitutions of equivalents may be made as circumstances may suggest or render expedient to cover various applications or implementations without departing from the spirit or the scope of the present disclosure. Further, it is to be understood that the phraseology and terminology employed herein are for the purpose of clarity of the description and should not be regarded as limiting.

Furthermore, in the present description, references to “one embodiment” or “an embodiment” means that a particular feature, structure, or characteristic described in connection with the embodiment is included in at least one embodiment of the present disclosure. The appearance of the phrase “in one embodiment” in various places in the specification is not necessarily refer to the same embodiment, nor are separate or alternative embodiments mutually exclusive of other embodiments. Further, the terms “a” and “an” used herein do not denote a limitation of quantity, but rather denote the presence of at least one of the referenced items. Moreover, various features are described which may be exhibited by some embodiments and not by others. Similarly, various requirements are described, which may be requirements for some embodiments but not for other embodiments.

The phrase “at least one of ‘A,’ ‘B,’ or ‘C’” means any possible combination of A, B, and C in a group or each A, B, or C alone. For example, “at least one of ‘A,’ ‘B,’ or ‘C’” may be interpreted as only A, only B, only C, only A and B, only A and C, only B and C, or A, B, and C altogether.

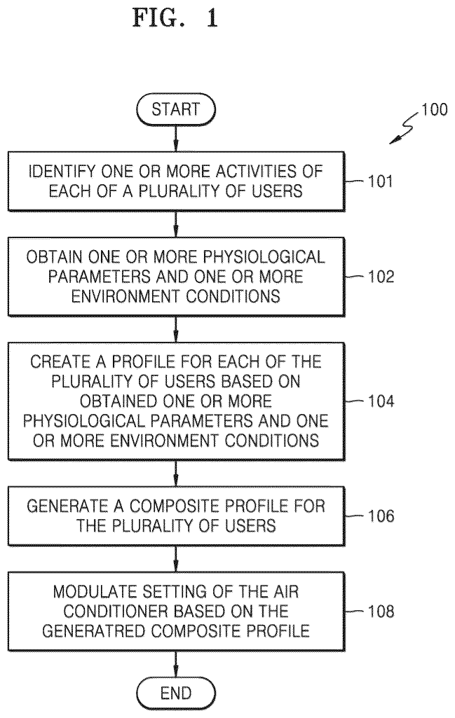

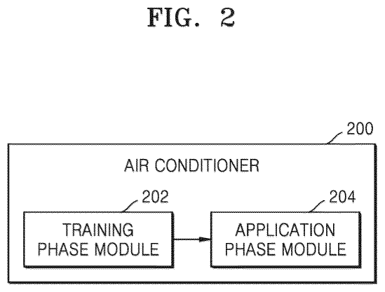

The device according to an embodiment of the invention as shown in FIG. 1 and FIG. 2 is utilized for adjusting the temperature of an indoor environment. In an exemplary embodiment, the device may be a temperature adjustment device, an air-conditioner, an electric fan, a climate-controlled system, an air cleaner, a cooling mat, and a heating device such as a heater, a boiler, a stove, and a heating mat. However, the present invention is not limited thereto and may be applied to the operation of other electronic devices that are capable of controlling the environment temperature. Referring to FIG. 1, a flow diagram showing a method (100) for enabling a bio-sleep functionality of a device in a multi-user environment is disclosed. The method may be explained in conjunction with the air conditioner disclosed in FIG. 2. In the flow diagram, each block may represent a module, segment, or portion of code, which comprises one or more executable instructions for implementing the specified logical function(s). It should also be noted that in some alternative implementations, the functions noted in the blocks may occur out of the order noted in the drawings. For example, two blocks shown in succession in FIG. 1 may be executed substantially concurrently or the blocks may sometimes be executed in the reverse order, depending upon the functionality involved. Any process descriptions or blocks in flowcharts should be understood as representing modules, segments, or portions of code that include one or more executable instructions for implementing specific logical functions or steps in the process, and alternate implementations are included within the scope of the example embodiments in which functions may be executed out of order from that shown or discussed, including substantially concurrently or in reverse order, depending on the functionality involved. In addition, the process descriptions or blocks in flow charts should be understood as representing decisions made by a hardware structure such as a state machine. The flow diagram starts at step 101 and proceeds to step 108.

At first, one or more activities of each of a plurality of users are identified, at step 101. Successively, one or more physiological parameters and one or more environment conditions are obtained, at step 102. In one embodiment, the one or more activities include active state and sleep state activity of one of the plurality of users, the one or more physiological parameters include at least one of heart rate, stress level, blood pressure, SPO2 level, or body temperature of one of the plurality of users and the one or more environment conditions include at least one of ambient temperature, humidity, or sleep timing pattern. Successively, a profile for each of the plurality of users is created, at step 104. In one embodiment, the profile is created based on at least one of the obtained one or more physiological parameters or one or more environment conditions. In one embodiment, the presence of the plurality of users could be detected. Thereafter, a composite profile for the plurality of users is generated, at step 106. Thereafter, the setting of the air conditioner based on the generated composite profile is modulated, at step 108. In one embodiment, the optimal setting of the device (e.g. the air conditioner) is enabled based on the generated composite profile.

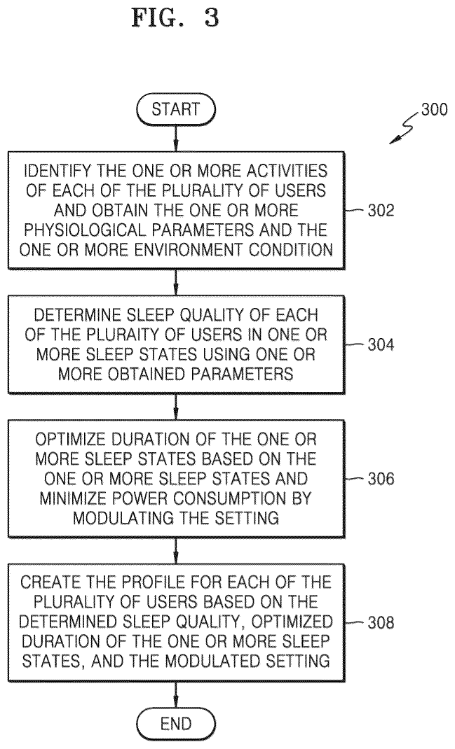

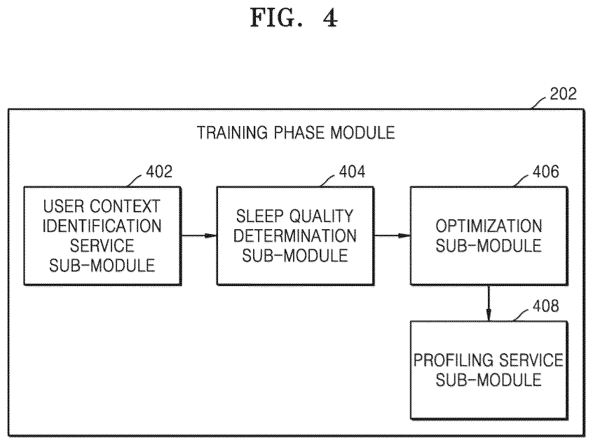

Referring to FIG. 2, a block diagram of an air conditioner 200 in a multi-user environment is illustrated, in accordance with one or more exemplary embodiments of the present disclosure. The air conditioner 200 may comprise a training phase module 202 configured for identifying one or more activities of each of plurality of users, obtaining one or more physiological parameters and one or more environment conditions, and creating a profile for each of the plurality of users based on at least one of the obtained one or more physiological parameters or one or more environment conditions. The training phase module 202 is described in more detail with reference to FIG. 3 and FIG. 4.

Referring to FIG. 3, a flow diagram showing a method of operation of the training phase module is illustrated, in accordance with one or more exemplary embodiments of the present disclosure. In the illustrated method 300, one or more activities of each of the plurality of users are identified and one or more physiological parameters are obtained, at step 302. In one embodiment, the one or more activities include active state and sleep state activity and the one or more physiological parameters include heart rate, body temperature, stress level, blood pressure, and SPO2 level of each of the plurality of users and one or more environment conditions include ambient temperature, humidity, and sleep timing pattern. Successively, the sleep quality of each of the plurality of users in one or more sleep states is determined, at step 304, using one or more obtained parameters. In one embodiment, the one or more obtained parameters include sleeping posture, sleep waking (the term “sleep-waking” and “urination” can be used interchangeably), dehydration, and frequency of change in the sleeping posture. Successively, duration of the one or more sleep states is optimized and power consumption is minimized, at step 306. In one embodiment, the duration of the one or more sleep states is optimized based on the one or more sleep states and power consumption is minimized by modulating the setting including air conditioning (ac) modes, fan speeds, ac temperature, etc. Successively, the profile is created, at step 308, for each of the plurality of users. In one embodiment, the profile of each of the plurality of users is created based on the determined sleep quality, optimized duration of the one or more sleep states, and the modulated setting. In another embodiment, the profile is updated with the sleep quality, optimized duration of the one or more sleep states and the modulated setting for each of the plurality of users.

Referring to FIG. 4, a block diagram of the training phase module 202 is illustrated, in accordance with one or more exemplary embodiments of the present disclosure. The training phase module 202 may include a user context identification service sub-module 402 for identifying the one or more activities such as active state and sleep state activity of each of the plurality of users and obtaining one or more physiological parameters of each of the plurality of users and one or more environment conditions such as ambient temperature, humidity, and sleep timing pattern. In one embodiment, the one or more activities of each of the user are determined from a wearable device through an electronic device. The wearable device may be, at least but not limited to, a wearable electronic device worn and/or implanted in the user's body, or tattooed on skin, wristband, and wristwatch and the electronic device may be, at least but not limited to, a mobile phone, PDA, ac remote control, any other handheld electronic device, and any other computing device. In another embodiment, the one or more activities of each of the users are determined directly from the electronic device.

On identifying the sleep activity of each of plurality of users and receiving the one or more physiological parameters from the wearable device, the device is set at a default ac temperature pre-stored in the memory of the device, based on the age of the user. In an exemplary embodiment, a sample dataset of default ac temperature is disclosed in table 1. Table 1 includes a sample dataset of default ac temperature. Table 1 includes different ac temperatures for different age groups. For a user of an age group in between 0 to 12, the device is set at an AC temperature ranging between 21 to 24 degree Celsius, for the age group between 12 and 19, the device is set at the AC temperature ranging between 17 and 19 degree Celsius and so on.

TABLE 1

| Age Group | AC Temperature (degrees Celsius) |
|---|---|
| 0-12 | 21-24 |
| 12-19 | 17-19 |
| 20-45 | 16-18.5 |
| 45-60 | 18-21 |
| 60+ | 22-24 |

The training phase module 202 further comprises a sleep quality determination sub-module 404 for determining sleep quality of each of the plurality of users in one or more sleep states using one or more derived parameters. In one embodiment, the one or more derived parameters include at least one of a sleeping posture, sleep waking, dehydration, or frequency of change in the sleeping posture. It should be noted that the sleep quality measures how well a user is sleeping or whether a user sleep is restful and restorative.

In one exemplary embodiment, Table 2 illustrates a sample dataset for the sleep quality determination considering the one or more physiological parameters, ac temperature, ac mode and the one or more derived parameters.

TABLE 2

| | | | | | | | | Frequency | | |
|---|---|---|---|---|---|---|---|---|---|---|
| | | | | | | | | of change | | Sleep |
| Sleeping | Body | Blood | SPO2 | AC | AC | Sleeping | Sleep | in sleeping | | Quality |
| State | Temp. | Pressure | Level | Temp. | Mode | Posture | waking | posture | Dehydration | Score |
| AWAKE | 38 | 85/126 | 97 | 18 | COOL | E | NO | 3 | NO | 0.82 |
| DSLEEP | 34 | 66/108 | 92 | 24 | SLEEP | B | YES | 1 | NO | 0.56 |
| REM | 32 | 62/103 | 91 | 22 | FAN | A | NO | 6 | YES | 0.18 |
| LSLEEP | 36 | 76/116 | 92 | 22 | DRY | D | YES | 2 | YES | 0.71 |
| DSLEEP | 33 | 67/105 | 88 | 22 | FAN | E | YES | 5 | NO | 0.31 |

As shown in Table 2, the sleep quality is determined by computing sleep quality score for each of the one or more sleep states based on ac temperature, ac mode, and the one or more derived parameters including sleeping posture, sleep waking (urination), dehydration, and frequency of change in the sleeping posture and the one or more physiological parameters. In one embodiment, the sleep quality score is computed using regression analysis. The regression analysis is used to predict value of a dependent variable based on the value of at least one independent variable and explaining impact of changes of the independent variable on the dependent variable. In one embodiment, the dependent variable is sleep quality score and independent variables include at least one of one or more physiological parameters, ac temperature, ac mode, and the one or more derived parameters. As shown in Table 2, nine independent variables are used for calculating the dependent using Equation 1 given below:

$$y = b 0 + b 1 * x 1 + b 2 * x 2 + bn * xn + ε i \qquad Equation 1$$

Wherein y is a dependent variable, which gives a sleep quality score; b0 is a population Y-intercept, b1 is a population slope coefficient; x1 is an independent variable; ci is a random error component.

For minimizing the sum of the squared differences between Y and Y′, where b0 and b1 are modified based on the loss (J) and change using gradient descent.

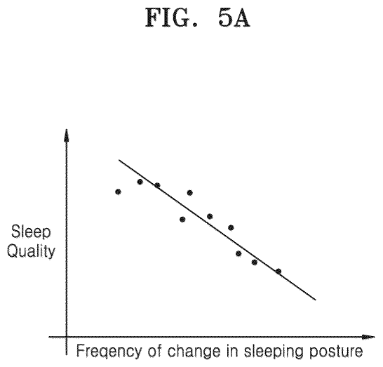

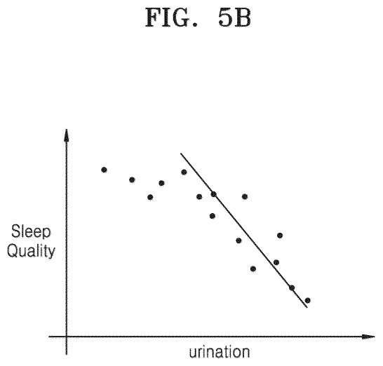

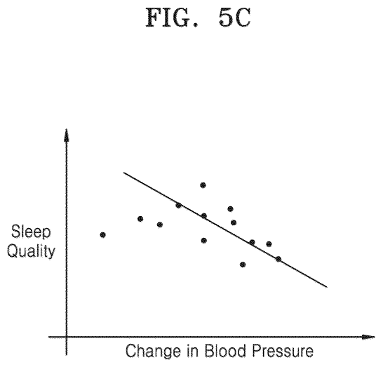

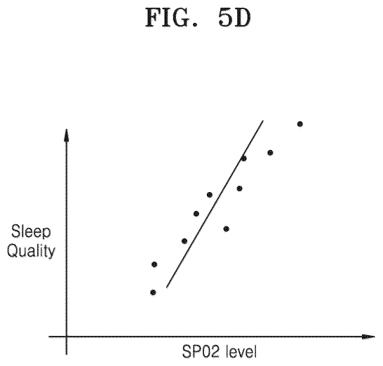

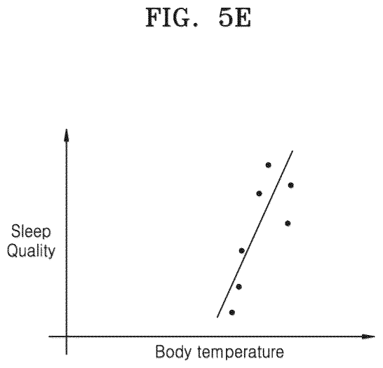

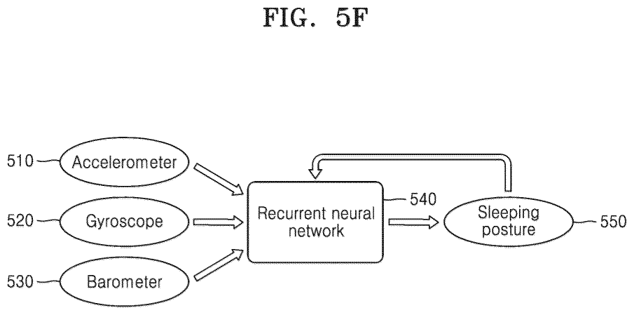

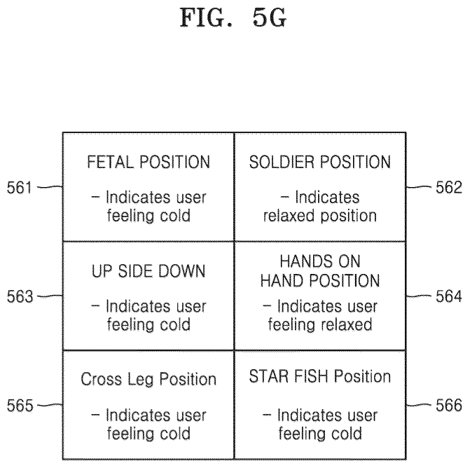

In one embodiment, the sleep quality score varies from 0 to 1 and represents sleep quality of each of the plurality of users, 0 represents discomfort sleep and 1 represents comfort sleep. Further, the sleep quality varies with respect to the derived parameters as illustrated in FIGS. 5a to 5e. According to FIG. 5a, the sleep quality varies inversely with respect to the frequency of change in the sleeping posture. According to FIG. 5b, the sleep quality varies inversely with respect to sleep waking (urination). According to FIG. 5c, the sleep quality varies inversely with respect to change in blood pressure. According to FIG. 5d, the sleep quality varies directly with respect to the SPO2 level. According to FIG. 5e, the sleep quality varies directly with respect to the body temperature.

The sleeping posture is a body configuration assumed by the user during or prior to sleeping. In one embodiment, the sleeping posture is used for identifying whether the user is sleeping comfortably or not. It should be noted that the sleeping posture has some health implications. The sleeping posture of each of the plurality of users is computed by utilizing a recurrent neural network (540). In one embodiment, inputs received from an accelerometer (510), a gyroscope (520), and a barometer (530) at different time stamps are fed to the recurrent neural network (540) one at a time to derive the sleeping posture (550) as illustrated in FIG. 5f. Equation 2 is used for deriving the sleeping posture (550):

$${h}_{t} = {f}_{w} ( {h}_{t - 1} , {x}_{t} ) = tanh ( {W}_{h h} {h}_{t - 1} + {W}_{xh} {x}_{t} ) , {y}_{t} = {W}_{h y} {h}_{t} \qquad Equation 2$$

Wherein, hₜ is new state at time t; f_{w} is same function with parameters W; hₜ₋₁ is an old state; xₜ is input vector at some time step; tanh is an activation function; yₜ is final output; Wₕₕ is weight at the recurrent neuron; and Wₓₕ is weight at the input neuron.

Wherein, activation function tanh (x) is computed using Equation 3 given below:

$$tanh ( x ) = 2 / ( 1 + e \hat{} - 2 x ) - 1 \qquad Equation 3$$

Different sleeping postures are known and they are fetal position 561, soldier position 562, upside down 563, hands on head position 564, cross leg position 565, star fish position 566, etc., which are illustrated in FIG. 5g, in accordance with one or more exemplary embodiments of the present disclosure.

The sleep-waking (urination) represents how often the user wakes up during sleep. Frequent waking at a sleeping time can disrupt the user's sleep cycle and reduce the sleep quality. Waking up once or not at all shows good sleep quality. Nocturia is a condition in which the user wakes up during the night because the user has to urinate a number of times which degrades sleep quality. Further, cold weather/environment also results in more urination as the body needs to filter more blood than normal as a greater volume of blood rushes to vital organs at a higher frequency.

In one embodiment, two factors such as blood pressure and stress level are used to classify the onset of urination. In an exemplary embodiment, the difference in systolic blood pressure for the user could be 4.2+10.7 mm Hg (P<0.001), and the difference in diastolic blood pressure could be 2.8+7.7 mm Hg (P<0.001) between holding urine and immediately after urination. Further, the stress causes muscle tension, which affects bladder muscles and increases the urge to urinate. In one embodiment, anxiety and depression are also associated with nocturia. The nocturia is a term used for frequently waking during sleep to urinate.

The training phase module 202 further comprises an optimization sub-module 406 for optimizing the duration of the one or more sleep states based on the one or more sleep states and minimizing power consumption by modulating device settings including ac modes, fan speeds, and ac temperature, etc. It should be noted that controlling the air conditioner (e.g. enabling bio-sleep functionality) is used to provide a comfortable time in bed and to reduce energy consumption at the same time. Therefore, a race condition always exists between AC power settings (ac temperature, fan speed, and cooling mode) and comfortable sleep so that both of these objectives can be achieved at the same time. Table 3 includes ace condition and respective objective.

TABLE 3

| S. No | Race Condition | Objective |
|---|---|---|
| 1 | AC Temperature | Increase for minimum power |
| | | consumption |
| 2 | Cooling Mode | Optimal |
| 3 | Awake State | Decrease Duration |
| 4 | Light Sleep State | Decrease Duration |
| 5 | Deep Sleep State | Increase Duration |
| 6 | REM State | Increase Duration |
| 7 | Sleep Quality | Enhance Sleep Quality |

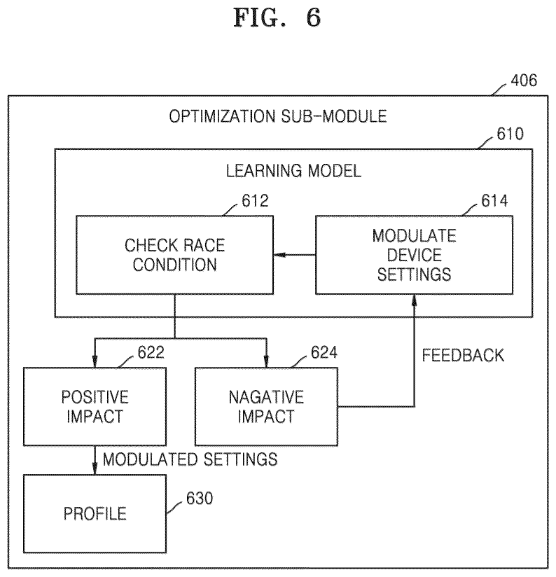

Referring to FIG. 6, a block diagram of the optimization sub-module 406 is illustrated, in accordance with one or more exemplary embodiments of the present disclosure. The optimization sub-module 406 includes a learning module 610 which utilizes reinforcement learning for optimizing the duration of the one or more sleep states and minimizing power consumption. In one embodiment, the optimization sub-module (406) checks a race condition 612 for the one or more objectives as disclosed in Table 3 and returns either a positive impact 622 or a negative impact 624. Table 4 discloses different impacts based on sleep comfort showing the duration of the one or more sleep states and power efficiency.

TABLE 4

| Power Efficiency | Sleep Comfort | Impact |
|---|---|---|
| Positive | Positive | Positive |
| Positive | Negative | Negative |
| Negative | Positive | Positive |
| Negative | Positive | Positive |
| Positive | No Effect | Positive |
| Negative | No Effect | Negative |

In case the impact is positive the modulated device settings are stored in the profile 630 of each of the plurality of users, else a feedback is sent to modulate the device settings 614 again.

The training phase module 202 further comprises a profiling service sub-module 408 for storing determined sleep quality and optimized duration of the one or more sleep states in the profile for each of the plurality of users. The profile includes one or more physiological parameters of each of the plurality of users, one or more environment conditions, the one or more derived parameters, and the device setting of each of the plurality of users. In one embodiment, the profile is created for each new user of the plurality of users. In another embodiment, the profile is updated for each existing user of the plurality of users.

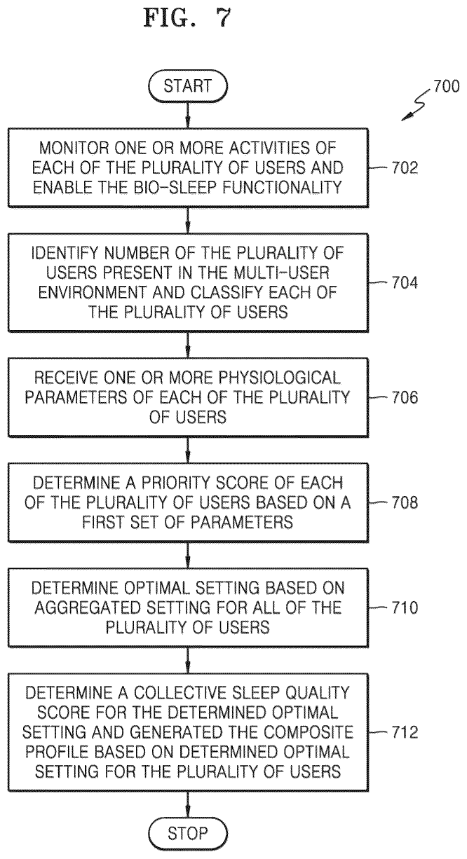

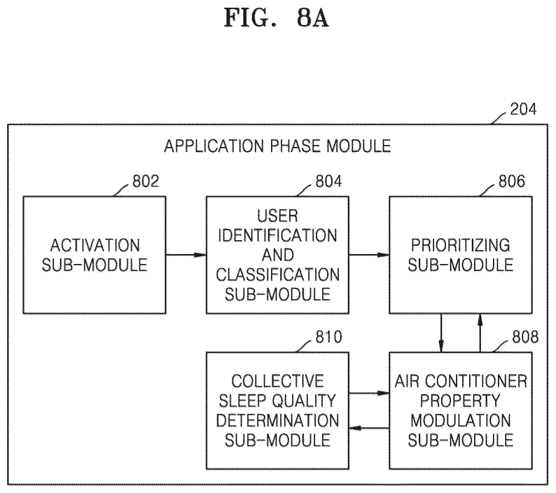

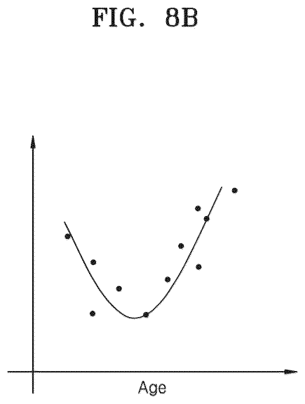

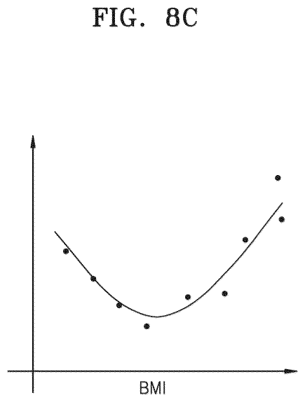

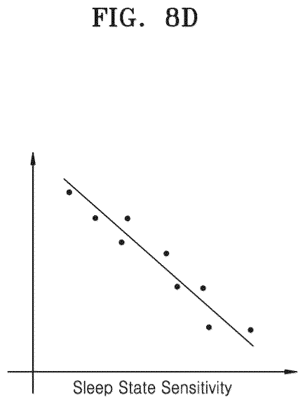

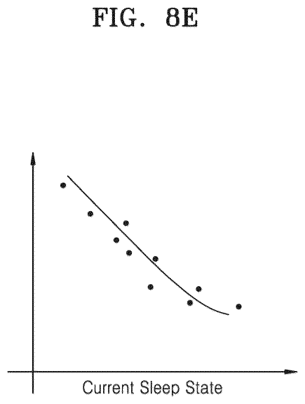

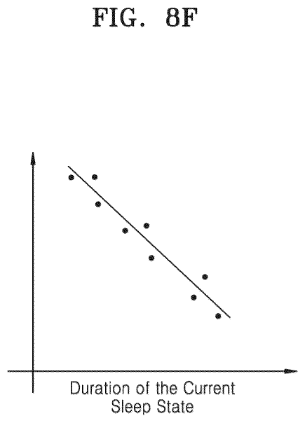

The air conditioner 200 further comprises an application phase module 204 configured for generating a composite profile for the plurality of users and modulating setting of the air conditioner based on the generated composite profile. The application phase module 204 is described in more detail with reference to FIG. 7 and FIG. 8a.

Referring to FIG. 7 a flow diagram showing a method of generating a composite profile by an application phase module in the multi-user environment is illustrated, in accordance with one or more exemplary embodiments of the present disclosure.

In the illustrated method 700, one or more activities of each of the plurality of users are monitored and bio-sleep functionality is enabled, at step 702. In one embodiment, the bio-sleep functionality is enabled based on one or more physiological parameters such as blood viscosity, blood pressure, SPO2 level, and body temperature of one of the plurality of users, the one or more activities of each of plurality of users monitored by the device, current environment condition such as humidity and status of the device such as backup power connection.

Successively, identification of the number of the plurality of users present in the multi-user environment and classification of each of the plurality of users into one of two categories is performed, at step 704. In one embodiment, the two categories include users with wearable devices and users without wearable devices.

Successively, one or more physiological parameters of each of the plurality of users are received, at step 706. The one or more physiological parameters includes at least blood pressure, body temperature, blood viscosity, and SPO2 level of each of the plurality of users. In one embodiment, the one or more physiological parameters of the users with wearable devices are received from wearable devices, and of the users without wearable devices are received from the users.

Successively, a priority score of each of the plurality of users based on the first set of parameters is determined, at step 708. The variation of the priority score of each of the plurality of users with respect to the first set of parameters is explained in FIGS. 8b to 8f. The first set of parameters include age, body mass index (BMI), sleep state sensitivity, current sleep state, and duration of the current sleep state. FIG. 8b illustrates a graphical representation of the priority score with respect to the age, in accordance with one or more exemplary embodiments of the present disclosure. FIG. 8c illustrates a graphical representation of the priority score with respect to the body mass index (BMI), in accordance with one or more exemplary embodiments of the present disclosure. FIG. 8d illustrates a graphical representation of the priority score with respect to the sleep state sensitivity, in accordance with one or more exemplary embodiments of the present disclosure. FIG. 8e illustrates a graphical representation of the priority score with respect to the current sleep state, in accordance with one or more exemplary embodiments of the present disclosure. FIG. 8f illustrates a graphical representation of the priority score with respect to the duration of the current sleep state, in accordance with one or more exemplary embodiments of the present disclosure.

Successively, an optimal setting based on aggregated setting for all of the plurality of users is determined, at step 710.

Successively, a collective sleep quality score based on the sleep quality score of each of the plurality of users for the determined optimal setting is determined, the optimal setting is enabled, and the composite profile is generated, at step 712. In one embodiment, the sleep quality score of each of the plurality of users is based on the ac temperature, ac mode and the one or more derived parameters including sleeping posture, sleep waking (urination), dehydration, and frequency of change in the sleeping posture and the one or more physiological parameters. The optimal setting of the device is enabled on determining the highest collective sleep quality score, and the composite profile is generated based on determined optimal setting of the device for the plurality of users

Referring to FIG. 8a, a block diagram of the application phase module 204 is illustrated, in accordance with one or more exemplary embodiments of the present disclosure. The application phase module 204 comprises an activation sub-module 802 configured for monitoring one or more activities such as the active state and sleep state activity of each of plurality of users and further configured for enabling the bio-sleep functionality. In one exemplary embodiment, Table 5 discloses a sample dataset for the activation sub-module 802.

TABLE 5

| Body | Blood | | One or more | Backup | |
|---|---|---|---|---|---|
| temperature | viscosity | Humidity | Activities | Power | Output |
| 37.5° C. | 3.3 | 49% | Active | ON | 1 |
| 36.3° C. | 4.7 | 40% | Active | OFF | 0 |
| 35.6° C. | 5.6 | 48% | Sleep | ON | 1 |
| 35.1° C. | 4.2 | 18% | Active | OFF | 1 |
| 37.0° C. | 5.5 | 25% | Sleep | OFF | 0 |
| 36.8° C. | 3.5 | 50% | Sleep | ON | 1 |
| 38.0° C. | 3.4 | 12% | Sleep | OFF | 1 |

Table 5 illustrates different values for one or more physiological parameters, current environment condition, one or more activities, status of backup power connection, and respective output.

The one or more physiological parameters such as body temperature measures how well a body can make and get rid of heat. The body generally maintains its temperature within a safe range, even when the temperature outside the body varies a lot. When the body is too hot, blood vessels in the skin widen to carry excess heat to the skin's surface. Any abnormal change in the body temperature is an indication of user discomfort, illness, fever, and blood viscosity which is a measure of the resistance of blood to flow. It is also described as the thickness and stickiness of blood. When blood has low viscosity, it travels quickly without much difficulty. Viscous blood is thicker and travels more slowly. The blood viscosity and temperature have an inverse relationship with respect to each other which means low temperature increases blood viscosity and vice-versa.

The current environment condition includes the percentage of humidity in the environment, humidity refers to the amount of water vapor present in the air. Humidity can make it feel even hotter on a summer day and increase the chances of a rainstorm developing. It is an important parameter to understand because it affects both weather and climate as well as global climate change. Humidity also affects the indoor environment. The higher the humidity, the wetter it feels outside.

The one or more activities include an active state or sleeping state activity. Generally, the body regulates heat based upon the current state of the one or more activities Therefore, it is very important to regulate heat externally when internal heat regulation is abnormal. Abnormal heat regulation occurs either during an extreme workout e.g. active state or a complete state of rest e.g. sleeping activity.

The status of the backup power connection shows if the device is using backup power or the direct supply of power and output corresponding to the different values for representing the status of the bio-sleep functionality.

The output provides the status of the bio-sleep functionality, which depends on the body temperature and the blood viscosity of each of the plurality of users, the humidity of the environment, one or more activities of each of the plurality of users, and power backup status of the device, and is enabled when the value in the output is 1 and disabled when the value in the output is 0. In one embodiment, a decision tree-based approach is utilized for enabling the bio-sleep functionality. In one exemplary embodiment, a sum of fifty rule-based classifiers are used to train the dataset. The impurity of the dataset calculation is performed using Equation 4 given below:

$$Imp = a 2 + b 2 , ( a - \# of 0 s ) ( b -> \# of 1 s ) \qquad Equation 4$$

After calculating impurity for every column and every possible value in the respective column, the one with the maximum score is chosen as a splitting rule at a node.

The application phase module (204 further comprises a user identification and classification sub-module 804 coupled to the activation sub-module 802 and configured for identifying the number of the plurality of users present in the multi-user environment. In one embodiment, the user identification and classification sub-module 804 utilizes one or more sensors including a camera sensor and/or a motion sensor mounted on the device or using a number of electronic devices connected to the device using wireless technologies such as Bluetooth technology or Wi-Fi technology.

The camera sensor is a sensor that detects the number of the plurality of users from signals reflected by each of the plurality of users. The signals can be light or other electromagnetic radiation. The motion sensor, also termed as a motion detector, is an electronic device that uses a sensor to detect the number of the plurality of users. The two most commonly used motion sensors are an active ultrasonic sensor and a passive infrared sensor. The active ultrasonic sensor emits an ultrasonic sound wave that bounces off the plurality of users in the immediate vicinity and returns to the motion sensor. The active ultrasonic sensor determines distance by measuring the time between sending and receiving ultrasonic sound waves. The passive infrared sensor detects fluctuations in infrared energy. The passive infrared sensor detects the presence of each of the plurality of users by detecting a change in the temperature of a given area.

The user identification and classification sub-module 804 is further configured for classifying each of the plurality of users into one of two categories including users with wearable devices and users without wearable devices. The wearable devices are connected to the electronic device using one of the technologies such as Bluetooth technology or Wi-Fi technology. The user identification and classification sub-module 804 is further configured for receiving one or more physiological parameters such as blood pressure, body temperature, blood viscosity, and SPO2 level of each of the plurality of users from either the wearable device or users. In one embodiment, the one or more physiological parameters are received from the wearable devices and the users through the electronic device.

The application phase module 204 further comprises a prioritizing sub-module 806 for determining the priority score of each of the plurality of users based on the first set of parameters. In one exemplary embodiment, Table 6 discloses a sample dataset for the prioritizing sub-module 806).

TABLE 6

| | | | Sleep state | | Duration of | |
|---|---|---|---|---|---|---|
| | | | sensitivity | Current | current | |
| Scenario | Age | BMI | {0 to 1} | sleep state | sleep state | Priority |
| {A, B, C, | {48, 18, 25, | {24, 20, 29, | {0.1, 0.5, 0.3, | {L3, L1, L3, | {80, 50, 60, | {D, B, C, |
| D} | 36} | 30} | 0.8} | L0} | 40} | A} |
| {A, B, C} | {20, 21, 23} | {18, 20, 24} | {0.2, 0.5, 0.6} | {L3, L2, L1} | {70, 60, 50} | {C, B, A} |
| {A, B, C, | {30, 70, 43, | {23, 32, 21, | {0.2, 0.9, 0.6, | {L2, L0, L2, | {30, 40, 55, | {E, B, D, |
| D, E} | 54, 22} | 29, 35} | 0.3, 0.5} | L1, L0} | 65, 15} | A, C} |
| {A, B, C, | {30, 22, 35, | {25, 22, 18, | {0.2, 0.5, 0.3, | {L3, L1, L3, | {90, 40, 85, | {F, E, D, |
| D, E, F} | 24, 18, 55} | 23, 25, 30} | 0.6, 0.8, 0.9} | L1, L0, L0} | 45, 30, 25} | B, C, A} |

Table 6 discloses four different scenarios having a different sequence of priorities based on different values of the first set of parameters such as age, body mass index (BMI), sleep state sensitivity, current sleep state, and duration of the current sleep state for each of the scenarios. In the first scenario, as shown in the table 6, the sequence of priorities is “DBCA” for four users represented by “A”, “B”, “C”, and “D”. Similarly, a different sequence of priorities for the different number of the plurality of users is disclosed in the rest of three scenarios in the table 6.

The sleep state sensitivity refers to body response towards some external stimulus. It is basically a natural ability of each of the plurality of the users to handle disturbance and remain in a sleeping state. Each of the plurality of users responds to such stimulus differently. In one embodiment, the sleep state sensitivity varies from 0 to 1, the low value of the sleep state sensitivity represents the likelihood of sleep being easily disturbed is low and the high value of the sleep state sensitivity represents the likelihood of sleep being easily disturbed is high. In an exemplary embodiment, if the disturbance in a deep sleep state lasts between 3 to 15 seconds and it may bring the user into a light sleep state or awake state, it means the sleep state sensitivity of the user is low. In another exemplary embodiment for any particular sleep state, if user movement is high and still user remains in same state, it means the sleep state sensitivity of the user is high.

The sleep state sensitivity also depends upon the user's current sleep state. In one embodiment, there exist four types of sleep states one is awake, second is light sleep, third is deep sleep, and fourth is rapid eye movement sleep, these four current sleep state is represented using L0, L1, L2, and L3, respectively. In one exemplary embodiment, the user with high sleep state sensitivity for any sleep state is given high priority.

In one embodiment, the sleep state sensitivity is determined using a gradient boosting algorithm. In the gradient boosting algorithm, many weak learners are combined to bring one strong learner. In one embodiment, the weak learners are individual decision trees connected in series and each tree tries to minimize the error of the previous tree. Due to the serial connection, the gradient boosting decision algorithm is usually slow to learn, but highly accurate.

For determining the priority score of each of the plurality of users, the prioritizing sub-module 806 performs multiple regression attempts to explain a dependent variable using more than one independent variable, assuming that there are no major correlations between the independent variables. In one embodiment, the dependent variable is a priority score and independent variables include the first set of parameters such as age, body mass index (BMI), sleep state sensitivity, current sleep state, and duration of the current sleep state. In one embodiment, the dependent variable is calculated using Equation 5:

$$Yi = β o + β 1 xi 1 + β2 xi 2 + β 3 x i 4 + β 4 x i 5 + ε \qquad Equation 5$$

Wherein, Yi is dependent variable (priority score); xi1 is age; xi2 is BMI; xi3 is sleep state sensitivity; xi4 is current sleep state; and xi5 is duration of the current sleep state.

The variation of the priority score (Yi) with respect to the first set of parameters is illustrated in FIGS. 8b to 8f.

The application phase module 204 further comprises an air conditioner property modulation sub-module 808 for determining the optimal setting of the device based on aggregated device settings for all of the plurality of users. In one embodiment, the air conditioner property modulation sub-module 808 modulates the device setting for all of the plurality of users which includes a preferred ac mode from the ac modes that include a dry mode, fan mode, cool mode, and sleep mode, a preferred fan speed from fan speeds which include low, medium, and high speed, and preferred temperature which is the sum of the product of ac temperature received from the profile of each of the plurality of users and determined priority score of each of the plurality of users. The ac temperature is calculated from Equation 6 given below:

$$Y = P 1 x 1 + P 2 x 2 + P 3 x 3 + PnXn \qquad Equation 6$$

Wherein Pn is ambient temperature stored in the profile of each of the plurality of users and xi is weightage of the user profile according to the priority score.

The application phase module 204 further comprises a collective sleep quality determination sub-module 810 for determining a collective sleep quality score based on the sleep quality score of each of the plurality of users for the determined optimal setting. In one embodiment, the sleep quality score is based on the ac temperature, ac mode, and the one or more derived parameters including sleeping posture, sleep waking (urination), dehydration, frequency of change in the sleeping posture, and the one or more physiological parameters. In one exemplary embodiment, the collective sleep quality score is determined considering the mean of sleep quality score of each of the plurality of users. The collective sleep quality determination sub-module 810 is further configured for maintaining the optimal setting of the device on determining the highest collective sleep quality score. In case, the collective sleep quality score is determined as low, the collective sleep quality determination sub-module 810 sends feedback to the air conditioner property modulation sub-module 808 for modulating the device settings again and to the prioritizing sub-module 806 for changing the priority sequence until the collective sleep quality score is determined as the highest.

In one embodiment, the user context identification service sub-module 402, the sleep quality determination sub-module 404, the an optimization sub-module 406, the a profiling service sub-module 408, the activation sub-module 802, the user identification and classification sub-module 804, the prioritizing sub-module 806, the air conditioner property modulation sub-module 808, and the collective sleep quality determination sub-module 810 may be implemented through an artificial intelligence reinforcement learning. A function associated with AI may be performed through the non-volatile memory, the volatile memory, and one or more processors.

The one or more processors may include one or a plurality of processors. At this time, one or a plurality of processors may be a general purpose processor, such as a central processing unit (CPU), an application processor (AP), or the like, a graphics-only processing unit such as a graphics processing unit (GPU), a visual processing unit (VPU), and/or an AI-dedicated processor such as a neural processing unit (NPU).

The one or a plurality of processors control the processing of the input data in accordance with a predefined operating rule or artificial intelligence (AI) model stored in the non-volatile memory and the volatile memory. The predefined operating rule or artificial intelligence model is provided through training or learning.

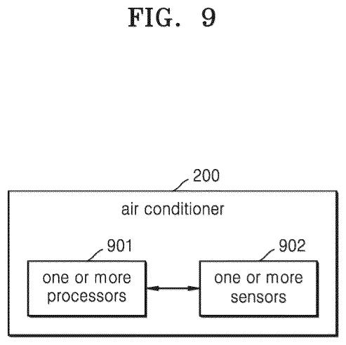

FIG. 9 illustrates the air conditioner 200 according to one or more embodiments. Accordingly, the air conditioner 200 may include one or more processors 901 and one or more sensors 902 communicable with each other.

Here, being provided through learning means that, by applying a learning algorithm to a plurality of learning data, a predefined operating rule or AI model of the desired characteristic is made. The learning may be performed in a device itself in which AI according to an embodiment is performed, and/or may be implemented through a separate server/system.

The AI model may consist of a plurality of neural network layers. Each layer has a plurality of weight values and performs a layer operation through the calculation of a previous layer and an operation of a plurality of weights. Examples of neural networks include, but are not limited to, convolutional neural network (CNN), deep neural network (DNN), recurrent neural network (RNN), restricted Boltzmann Machine (RBM), deep belief network (DBN), bidirectional recurrent deep neural network (BRDNN), generative adversarial networks (GAN), and deep Q-networks.

The learning algorithm is a method for training a predetermined target device (for example, a robot) using a plurality of learning data to cause, allow, or control the target device to make a determination or prediction. Examples of learning algorithms include, but are not limited to, supervised learning, unsupervised learning, semi-supervised learning, or reinforcement learning.

While example embodiments of the disclosure have been shown and described, the disclosure is not limited to the aforementioned specific embodiments, and it is to be understood that various modifications may be made by those having ordinary skill in the technical field to which the disclosure belongs, without departing from the gist of the disclosure as claimed by the appended claims. Further, it is intended that such modifications are not to be interpreted independently from the technical idea or prospect of the disclosure.

What is claimed is:

1. A method for controlling an air conditioner in a multi-user environment, the method comprising:
identifying, by one or more sensors, one or more activities of each of a plurality of users;
obtaining, by the one or more sensors, one or more physiological parameters of each of the plurality of users and one or more environment conditions;
creating, by one or more processors, a profile for each of the plurality of users based on at least one of the one or more physiological parameters or the one or more environment conditions;
generating, by the one or more processors, a composite profile for each of the plurality of users; and
modulating, by the one or more processors, a setting of the air conditioner based on the composite profile of each of the plurality of users.

2. The method according to claim 1, wherein the one or more activities comprise active state and sleep state activity of one of the plurality of users,
wherein the one or more physiological parameters comprise at least one of heart rate, stress level, blood pressure, SPO2 level, or body temperature of one of the plurality of users, and
wherein the one or more environment conditions comprise at least one of ambient temperature, humidity, or sleep timing pattern.

3. The method according to claim 1, wherein the creating the profile for each of the plurality of users comprises:
determining, by the one or more processors, sleep quality of each of the plurality of users in one or more sleep states using one or more derived parameters, wherein the one or more derived parameters comprise at least one of a sleeping posture, sleep waking, dehydration, or frequency of change in the sleeping posture;
optimizing, by the one or more processors, a duration of the one or more sleep states based on the one or more sleep states and minimizing power consumption by modulating the setting, wherein the setting comprises one or more air conditioning (ac) modes, fan speeds, and ac temperature; and
creating, by the one or more processors), the profile for each of the plurality of users based on at least one of the determined sleep quality, the optimized duration of the one or more sleep states, or the modulated setting.

4. The method according to claim 3, wherein the generating the composite profile for each of the plurality of users comprises:
monitoring, by the one or more sensors, the one or more activities of each of the plurality of users;
enabling, by the one or more processors, a bio-sleep functionality;
identifying, by the one or more sensors or one or more electronic devices, a number of the plurality of users present in the multi-user environment and classifying each of the plurality of users;
receiving, by the one or more processors, the one or more physiological parameters of each of the plurality of users;
determining, by the one or more processors, a priority score of each of the plurality of users based on a first set of parameters received from the profile;
determining, by the one or more processors, the setting of the air conditioner based on an aggregated setting for all of the plurality of users, wherein the aggregated setting comprises the setting for all of the plurality of users received from the profile;
determining, by the one or more processors, a collective sleep quality score based on a sleep quality score of each of the plurality of users for the setting for all of the plurality of users received from the profile;
enabling, by the one or more processors, the setting of the air conditioner based on determining a highest collective sleep quality score of the sleep quality score of each of the plurality of users; and
generating, by the one or more processors, the composite profile based on the setting for all of the plurality of users received from the profile.

5. The method according to claim 4, wherein the bio-sleep functionality is enabled based on the one or more physiological parameters including blood viscosity and body temperature of each of the plurality of users, the one or more environment conditions including humidity, and a status of the air conditioner including backup power connection.

6. The method according to claim 4, wherein the one or more sensors comprise at least one of a camera sensor or a motion sensor mounted on the air conditioner, wherein the one or more electronic devices are connected to the air conditioner via Bluetooth technology or Wi-Fi technology, and
wherein the method further comprises classifying, by the one or more processors, each of the plurality of users into including first category of users with wearable devices and a second category of users without wearable devices.

7. The method according to claim 6, wherein receiving the one or more physiological parameters of each of the plurality of users comprises:
receiving, from the wearable devices, the one or more physiological parameters of each of the plurality of users with the wearable devices; and
receiving, from electronic devices, the one or more physiological parameters of each of the plurality of users without the wearable devices, the electronic devices being at least one of a mobile phone, PDA, or ac remote control,
wherein the wearable devices include at least one of wearable electronic devices, electronic devices implanted in a user's body, wearable devices tattooed on skin, wristbands, or wristwatches, and
wherein the wearable devices are connected to the electronic devices by at least one of Bluetooth technology or Wi-Fi technology.

8. The method according to claim 4, wherein the first set of parameters comprise at least one of age, body mass index (BMI), sleep state sensitivity, current sleep state, or a duration of the current sleep state.

9. The method according to claim 4, wherein the aggregated setting comprises:
a preferred ac mode from one or more ac modes which comprising a dry mode, a fan mode, a cool mode, or sleep mode;
a preferred fan speed from one or more fan speeds comprising at least one of a low speed, a medium speed, or high speed; and
a preferred ac temperature being a sum of a product of the ac temperature received from the profile of each of the plurality of users and the priority score of each of the plurality of users.

10. The method according to claim 9, wherein the sleep quality score is based on the preferred ac temperature, the preferred ac mode, the one or more derived parameters, and the frequency of change in the sleeping posture and the one or more physiological parameters.

11. An air conditioner in a multi-user environment, comprising:
one or more sensors configured to:
identify one or more activities of each of a plurality of users; and
obtain one or more physiological parameters of each of the plurality of users and one or more environment conditions; and
one or more processors configured to:
create a profile for each of the plurality of users based on at least one of the one or more physiological parameters or the one or more environment conditions;
generate a composite profile for each of the plurality of users; and
modulate a setting of the air conditioner based on the composite profile of each of the plurality of users.

12. The air conditioner according to claim 11, wherein the one or more activities comprise active state and sleep state activity of one of the plurality of users, wherein the one or more physiological parameters comprise at least one of heart rate, stress level, blood pressure, SPO2 level, or body temperature of one of the plurality of users, and
wherein the one or more environment conditions comprise at least one of ambient temperature, humidity, or sleep timing pattern.

13. The air conditioner according to claim 11, wherein the one or more processors are further configured to:
determine sleep quality of each of the plurality of users in one or more sleep states using one or more derived parameters, wherein the one or more derived parameters comprise at least one of a sleeping posture, sleep waking, dehydration, or frequency of change in the sleeping posture;
optimize a duration of the one or more sleep states based on the one or more sleep states and minimize power consumption by modulating the setting, wherein the setting comprises one or more air conditioning (ac) modes, fan speeds, and ac temperature; and
create the profile for each of the plurality of users based on at least one of the determined sleep quality, optimized duration of the one or more sleep states, or the modulated setting.

14. The air conditioner according to claim 13, wherein the one or more sensors are further configured to monitor the one or more activities of each of plurality of users; and
wherein the one or more processors are further configured to:
enable a bio-sleep functionality;
identify a number of the plurality of users present in the multi-user environment, classifying each of the plurality of users;
receive the one or more physiological parameters of each of the plurality of users;
determine a priority score of each of the plurality of users based on a first set of parameters received from the profile;
determine the setting of the air conditioner based on an aggregated setting for all of the plurality of users, wherein the aggregated setting comprises the setting for all of the plurality of users received from the profile;
determine a collective sleep quality score based on a sleep quality score of each of the plurality of users for the setting for all of the plurality of users received from the profile;
enable the setting of the air conditioner based on a highest collective sleep quality score of the sleep quality score of each of the plurality of users; and
generate the composite profile based on the setting for all of the plurality of users received from the profile.

15. The air conditioner according to claim 14, wherein the one or more processors are further configured to enable the bio-sleep functionality based on the one or more physiological parameters including blood viscosity and body temperature of each of the plurality of users, the one or more environment conditions including humidity, and a status of the air conditioner including backup power connection.
